(19) United States
(12) Patent Application Publication
(10) Pub. No.: US 2025/0352369 A1
(43) Pub. Date: Nov. 20, 2025

(54) PSEUDO HOOKED WIRE STENT

(71) Applicant: BOSTON SCIENTIFIC SCIMED, INC., Maple Grove, MN (US)

(72) Inventors: GARY GILMARTIN, Foxford (IE); THOMAS MARTIN KEATING, Galway (IE); DANIEL TUCK, Galway (IE); FIONN STAPLETON, Galway (IE); JOHN THOMAS O'DRISCOLL, Galway (IE); CELINE GLYNN, Galway (IE); HARRY MULLEADY, Galway (IE)

(73) Assignee: BOSTON SCIENTIFIC SCIMED, INC., Maple Grove, MN (US)

(21) Appl. No.: 19/209,082

(22) Filed: May 15, 2025

Related U.S. Application Data
(60) Provisional application No. 63/648,491, filed on May 16, 2024.

(51) Int. Cl. A61F 2/88

(52) U.S. Cl. CPC A61F 2/885; A61F 2220/0016; A61F 2230/0017

(57) ABSTRACT

A stent is disclosed. The stent includes a tubular scaffold extending from a first end to a second end in which the tubular scaffold is formed of a single filament shaped to form a plurality of open cells throughout the tubular scaffold. Each of the open cells may be formed as a parallelogram shape defined by two pairs of opposing linear sections of the filament and pseudo hooked sections of the filament at each apex of the plurality of open cells, wherein each of apices of the plurality of open cells includes a pseudo hooked region in which first and second bends of the single filament overlap one another. The open cells may include first and second helical rows of small open cells, and a first helical row of large open cells positioned therebetween.

### CROSS-REFERENCE TO RELATED APPLICATIONS

The application claims the benefit of U.S. Provisional Patent Application Ser. No. 63/648,491, filed on May 16, 2024, the disclosure of which is incorporated herein by reference.

### TECHNICAL FIELD

The present disclosure pertains generally, but not by way of limitation, to medical devices and systems, and methods of treatment. More particularly, the present disclosure relates to stents, stent configurations, and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure. These devices are manufactured by any one of a variety of different manufacturing methods and may be used according to any one of a variety of methods for a variety of applications. Of the known medical devices, delivery systems, and methods, each has certain advantages and disadvantages. For example, in some stents, the compressible and flexible properties that assist in stent delivery may also result in a stent that has a tendency to migrate from its originally deployed position in a body lumen. There is an ongoing need to provide alternative medical devices and delivery devices as well as alternative methods for manufacturing and using medical devices and delivery devices, such as those susceptible to migration in the anatomy.

### BRIEF SUMMARY

This disclosure provides design, material, manufacturing method, and use alternatives for medical devices.

One example is a stent. The stent includes a tubular scaffold extending from a first end to a second end. The tubular scaffold is formed of a single filament. The single filament is shaped to form a plurality of open cells throughout the tubular scaffold, wherein each of the plurality of open cells defined by a plurality of apices of the single filament. At least one of the plurality of apices includes a pseudo hooked segment wherein a first bend of the single filament longitudinally overlaps a second bend of the single filament.

Alternatively or additionally to any of the examples herein, in another example, the plurality of open cells includes a first helical row of large open cells, a first helical row of small open cells, and a second helical row of small open cells. The first helical row of large open cells is positioned between the first helical row of small open cells and the second helical row of small open cells.

Alternatively or additionally to any of the examples herein, in another example, each of the large open cells has a greater perimeter and/or area than each of the small open cells.

Alternatively or additionally to any of the examples herein, in another example, each of the large open cells has a longitudinal extent greater than a longitudinal extent of each of the small open cells.

Alternatively or additionally to any of the examples herein, in another example, each of the large open cells has a parallelogram shape having four apices.

Alternatively or additionally to any of the examples herein, in another example, each of the four apices of the large open cells includes a pseudo hooked segment.

Alternatively or additionally to any of the examples herein, in another example, each of the small open cells has a rhombus shape having four apices.

Alternatively or additionally to any of the examples herein, in another example, each of the four apices of the small open cells includes a pseudo hooked segment.

Another example is a stent. The stent includes a tubular scaffold extending from a first end to a second end. The tubular scaffold is formed of a single filament defining a plurality of open cells having apices. The single filament forms a plurality of circumferential rings, wherein each circumferential ring is formed of the single filament in which the single filament undulates to form a plurality of peaks oriented toward the first end and a plurality of valleys oriented toward the second end. Each of the apices of the plurality of open cells includes a pseudo hooked region in which the plurality of peaks of a first circumferential ring of the plurality of circumferential rings longitudinally overlap the plurality of valleys of a second circumferential ring of the plurality of circumferential rings.

Alternatively or additionally to any of the examples herein, in another example, in an axially elongated, non-equilibrium configuration, the plurality of peaks of the first circumferential ring are axially spaced apart from the plurality of valleys of the second circumferential ring with an axial gap therebetween.

Alternatively or additionally to any of the examples herein, in another example, each of the plurality of open cells is formed as a parallelogram shape defined by two pairs of opposing linear sections of the filament and pseudo hooked regions of the filament at each apex of the plurality of open cells.

Alternatively or additionally to any of the examples herein, in another example, the plurality of open cells includes a first helical row of large open cells, a first helical row of small open cells, and a second helical row of small open cells. The first helical row of large open cells is positioned between the first helical row of small open cells and the second helical row of small open cells.

Alternatively or additionally to any of the examples herein, in another example, each of the large opens cells shares a side with one of the small open cells.

Alternatively or additionally to any of the examples herein, in another example, each of the large open cells has a longitudinal extent greater than a longitudinal extent of each of the small open cells.

Another example is a stent. The stent includes a tubular scaffold extending from a first end to a second end. The tubular scaffold is formed of a single filament. The single filament is shaped to form a plurality of open cells throughout the tubular scaffold. Each of the plurality of open cells is defined by at least two pairs of opposing linear sections of the single filament and at least two pseudo hooked segments of the single filament. The single filament is bent to form the at least two pairs of opposing linear sections and the at least two pseudo hooked segments. A first bend of the single filament longitudinally overlaps a second bend of the single filament at each pseudo hooked segment.

Alternatively or additionally to any of the examples herein, in another example, each pseudo hooked segment includes a peak of the single filament in a first circumferential ring of the tubular scaffold longitudinally overlapping a valley of the single filament in a second circumferential ring of the tubular scaffold.

Alternatively or additionally to any of the examples herein, in another example, the plurality of open cells are arranged in a plurality of helical rows extending helically around the tubular scaffold, wherein at least one helical row of the plurality of helical rows comprises a plurality of open cells with a larger perimeter than the plurality of open cells of at least one other helical row of the plurality of helical rows.

Alternatively or additionally to any of the examples herein, in another example, each of the plurality of open cells has a parallelogram shape having four apices.

Alternatively or additionally to any of the examples herein, in another example, each of the four apices of the open cells includes a pseudo hooked segment.

Alternatively or additionally to any of the examples herein, in another example, each of the plurality of open cells conforms to a parallelogram shape.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative stent;

FIG. 2 is a schematic view of the tubular scaffold of the stent of FIG. 1;

FIG. 3 is a side view of the illustrative stent of FIG. 1, where the tubular scaffold has been longitudinally stretched;

FIG. 4 is a side view of the illustrative stent of FIG. 1, where the stent includes a covering; and

FIG. 5 is a side view of the illustrative stent of FIG. 1 where the stent has been constrained in a curved configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Endoscopic retrograde cholangiopancreatography (ERCP) is primarily used to diagnose and treat conditions of the bile ducts, including, for example, gallstones, inflammatory strictures, leaks (e.g., from trauma, surgery, etc.), and cancer. Through the endoscope, the physician can see the inside of the stomach and the duodenum, and inject dyes into the ducts in the bile tree and pancreas so they can be seen on X-rays. These procedures may necessitate gaining and keeping access to the biliary duct, which may be technically challenging, may require extensive training and practice to gain proficiency, and may require one or more expensive tools in order to perform. Blockage of the biliary duct may occur in many of the disorders of the biliary system, including the disorders of the liver, such as, primary sclerosing cholangitis, stone formation, scarring in the duct, etc. This requires the need to drain blocked fluids from the biliary system, to treat the disorders.

During an ERCP procedure, a number of steps are typically performed while the patient is often sedated and anaesthetized. For example, an endoscope may be inserted through the mouth, down the esophagus, into the stomach, through the pylorus into the duodenum, to a position at or near the ampulla of Vater (the opening of the common bile duct and pancreatic duct). Due to the shape of the ampulla and the angle at which the common bile and pancreatic ducts meet the wall of the duodenum, the distal end of the endoscope is generally placed just past the ampulla. Due to positioning of the endoscope beyond the ampulla, the endoscopes used in these procedures are usually side-viewing endoscopes. The side-viewing feature provides imaging along the lateral aspect of the tip rather than from the end of the endoscope. This allows the clinician to obtain an image of the medial wall of the duodenum, where the ampulla of Vater is located, even though the distal tip of the endoscope is beyond the opening.

Applying a stent to a duct of the biliary tree may reduce obstructions and enable the duct (e.g., a bile duct and/or other suitable duct) to remain patent (e.g., open) in a presence of a stricture. When the stent is deployed from a delivery catheter, the stent radially expands and keeps the lumen patent, which may facilitate bile drainage through the duct.

Although embodiments of the present disclosure are described with specific reference to medical devices (e.g., stents) and systems for restriction or drainage of the gallbladder, pseudocysts, gastrojejunostomy, and/or the like, it should be appreciated that such medical devices may be used in a variety of medical procedures (e.g., external biliary drain conversion, enteroenterostomy, gastroduodenostomy and gastroilcostomy, etc.) to establish and/or maintain a temporary or permanent restriction or open flow passage from, along, or between a variety of body organs, lumens, ducts, vessels, fistulas, cysts and spaces (e.g., the dermis, stomach, duodenum, jejunum, small intestine, gallbladder, kidneys, pancreas, biliary trees, pancreatic trees, bladder, ureter, abscesses, walled-off pancreatic necrosis, bile ducts, etc.). The devices may be inserted via different access points and approaches, e.g., percutaneously, endoscopically, laparoscopically or some combination thereof. The medical devices disclosed herein are self-expanding, but in other embodiments the medical devices may be expandable by other means, including, e.g., a balloon catheter. Moreover, such medical devices are not limited to restriction or drainage, but may facilitate access to organs, vessels, or body lumens for other purposes, such as creating a path to divert or bypass fluids or solids from one location to another, removing obstructions and/or delivering therapy, including non-invasive or minimally invasive manipulation of the tissue within the organ and/or the introduction of pharmacological agents via the open flow passage.

Stent deployment may be effected in any suitable manner. In some examples, stent deployment may include delivering a stent in a distal end of a delivery system (e.g., a co-axial delivery system and/or other suitable delivery system) to a target location or site within a patient (e.g., at a location of a biliary stricture and/or other suitable location), positioning a proximal handle of a delivery device against a chest or stomach of a practitioner (e.g., a physician, nurse, etc.), and pulling on a distal handle in a proximal direction towards the proximal handle. Pulling the distal handle in the proximal direction may slide a sheath (e.g., any suitable external tube, which may be known as an e-tube) covering the stent proximally to expose the stent while maintaining a position of an inner elongate member at the target location or site. As the sheath is withdrawn from the stent, the stent radially expands and shortens lengthwise (e.g., the stent foreshortens in a proximal direction). As a result of the shortening of the stent, the practitioner must consider an expected shortening (e.g., shortening in the proximal direction and/or other shortening) of the stent when positioning the stent and delivery device at the target location or site, which may result in poor alignment of the stent with the target location or site (e.g., relative to a location of a target stricture, etc.) and/or needing to re-position the stent after initial deployment.

Shortening of a stent during deployment may occur with stents having braided, knitted or overlapping structure. Stents formed by laser cutting a monolithic piece of material (e.g., a hypotube) may be less prone to shortening upon deployment than stents having a braided, knitted, or overlapping structure and as such, may provide practitioners with increased control over positioning of a stent across the target location or site relative to the control provided when using a braided, a knitting, or overlapping structure. Further, stents of a laser cut construction may have a lower constrained diameter (e.g., diameter when in the delivery device) relative to a constrained diameter of braided, knitted, or overlapping stents, which may facilitate delivering the stent to small diameter ducts, such as hepatic and/or other biliary ducts, using a small diameter delivery device (e.g., having a 6F diameter and/or another suitable diameter). In some cases, such a lower constrained diameter and a small diameter delivery device may facilitate dual stenting of the hepatic and/or other biliary ducts, where two delivery devices are placed through an endoscope to a target location or site and are used to deploy the stents simultaneously.

However, stents having a laser cut construction have drawbacks. For example, stents of a laser cut construction are often bare or uncovered, which results in tissue ingrowth at and/or around the stent that makes removal of the stent after a period of time difficult or impossible without injuring the patient. In another example, stents having a laser cut construction cannot be re-constrained after at least partial deployment during placement, which may complicate positioning the stent at the target location or site.

The stent configurations discussed herein may be configured to have a small constrained diameter and mitigate foreshortening during deployment of the stent. Additionally, the stent configurations discussed herein may be configured 1) to facilitate being re-constrained after at least partial deployment and 2) to be covered and/or coated to prevent or mitigate tissue ingrowth after initial deployment.

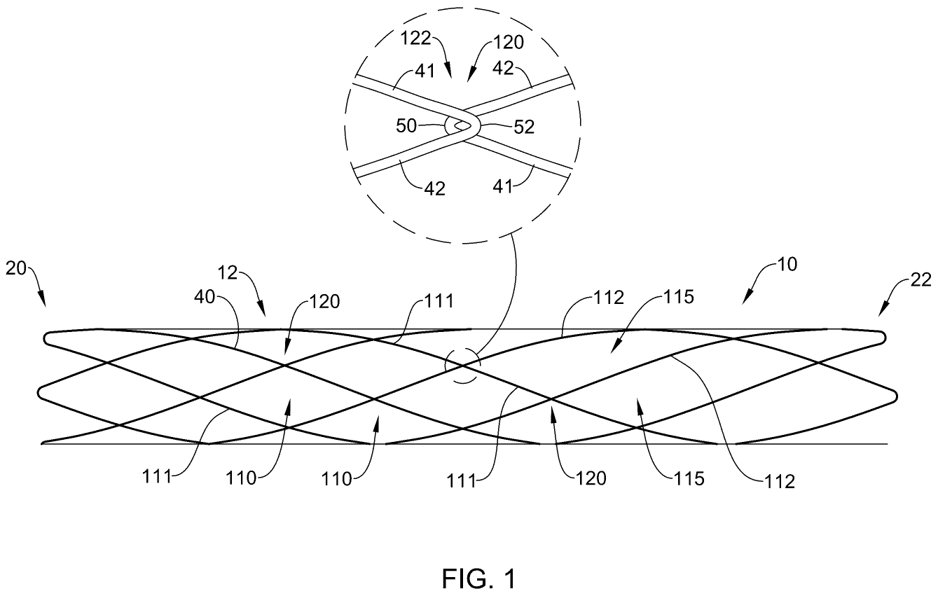

FIG. 1 depicts a side view of a stent 10 according to examples of the present disclosure. In this and other examples, stent 10 includes a tubular scaffold 12 having a first end 20, a second end 22, and a body extending therebetween. The tubular scaffold 12 may define a lumen extending through the stent 10 from the first end 20 to the second end 22. The tubular scaffold 12 may be formed from a single filament 40, and the single filament 40 may be shaped to form a plurality of open cells 110, 115 throughout the body of the stent 10. Each of the plurality of open cells 110, 115 may include opposing linear sections 111, 112 and apices 120 where two adjacent linear sections 111, 112 converge. As shown in the enlarged portion of FIG. 1, the apices 120 may be locations where the filament 40 includes a pair of bends that are longitudinally overlapped with one another to form a pseudo hooked section 122. As used herein, “pseudo hooked section” is intended to refer to a location of the tubular scaffold 12 in which a bend or peak of the single filament 40 longitudinally overlaps with another bend or valley of the single filament, yet the bends are not intertwined, twisted, hooked or otherwise interlocked with one another. Thus, because the peak (i.e., a first bend) of the single filament longitudinally overlaps with the valley (i.e., a second bend) of the single filament, the bend or peak of the single filament appears to be interlocked with the second bend or valley of the single filament when viewed from the side of the stent 10; however the first bend (i.e., peak) is not actually interlocked with the second bend (i.e., valley) at the pseudo hooked section.

As shown in the enlarged portion of FIG. 1, the filament 40 may bend at a peak 50 and change direction at the bends such that a first segment 41 of the filament 40 on a first side of a bend or peak 50 extends in a first helical direction from the bend or peak 50 at the pseudo hooked section 122 and a second segment 42 of the filament 40 on a second side of the bend or peak 50 extends in a second helical direction from the bend at the pseudo hooked section 122. Both the first segment 41 and the second segment 42 of the filament 40 may extend toward the same end (e.g., the second end 22) from the bend or peak 50. Thus, the peaks 50 may all be bends in the filament 40 oriented toward the first end 20. The other bend in the filament 40 overlapped at the pseudo hooked section 122 can be similarly formed, with its first and second segments 41, 42 each extending toward the opposite end (e.g., the first end 20) from the bend or valley 52. Thus, the filament 40 may bend at a valley 52 and change direction at the bends such that a first segment 41 of the filament 40 on a first side of a bend or valley 52 extends in a first helical direction from the bend or valley 52 at the pseudo hooked section 122 and a second segment 42 of the filament 40 on a second side of the bend or valley 52 extends in a second helical direction from the bend at the pseudo hooked section 122. Both the first segment 41 and the second segment 42 of the filament 40 may extend toward the same end (e.g., the first end 20) from the bend or valley 52. Thus, the valleys 52 may all be bends in the filament 40 oriented toward the second end 22.

Accordingly, the tubular scaffold 12 may be formed of a plurality of circumferential rings in which the filament 40 undulates to form a plurality of peaks 50 and valleys 52 between first segments 41 and second segments 42 of the filament 40. Thus, the peaks 50 of the filament 40 in a first undulating circumferential row of the filament 40 may overlap with the valleys 52 of the filament 40 in a second undulating circumferential row of the filament 40, the peaks 50 of the filament 40 in the second undulating circumferential row of the filament 40 may overlap with the valleys 52 of the filament 40 in a third undulating circumferential row of the filament 40, the peaks 50 of the filament 40 in the third undulating circumferential row of the filament 40 may overlap with the valleys 52 of the filament 40 in a fourth circumferential row of the filament 40, and so on.

In some embodiments, the apices 120 of the open cells (110, 115) may include at least two pseudo hooked sections 122. In some instances, each of the apices 120 of the open cells (110, 115) may include a pseudo hooked section 122. In other words, in some instances each open cell 110, 115 may include four apices 120, with each apex 120 formed of a pseudo hooked section 122 of the filament 40. Accordingly, each of the intersections of the filament 40 shown in FIG. 1 may be a pseudo hooked section 122 in which a peak 50 of the filament 40 overlaps with a valley 52 of the filament 40 (e.g., one of the peak 50 and valley 52 is positioned radially outward of the other of the peak 50 and valley 52), yet not interlocked with one another.

Opposing pairs of the linear sections 111, 112 of each of the plurality of open cells 110, 115 may be in parallel or substantially parallel alignment with one another. In other words, the linear sections 111, 112 on opposite sides of the open cells 110, 115 that form each of the plurality of open cells 110, 115 may be parallel or substantially parallel to one another. Thus, each open cell 110, 115 may be defined by two pairs of opposing linear sections 111, 112 on opposite sides of the open cells 110, 115. Accordingly, the opposing linear sections 111, 112 of each of the plurality of open cells 110, 115 may be spaced apart from one another, on opposite sides of each of the plurality of open cells 110, 115. In this and other examples, the two opposing pairs of linear sections 111, 112 and the pseudo hooked sections 122 (e.g., four pseudo hooked sections 122) of each of the plurality of open cells 110, 115 form a perimeter of the open cell 110, 115 that is constructed of the single filament 40. The perimeter of the open cells 110, 115 may be defined as the combined length of the single filament 40 segments which forms the boundaries of each of the open cells 110, 115. The area of the open cells 110, 115 may be defined as the area contained within the perimeter (i.e., the boundary created by weaving of the single filament 40) of the open cells 110, 115. The perimeter of one or more of the open cells 110, 115 of the plurality of open cells 110, 115 may conform to various geometries and shapes.

The perimeter of one or more of the open cells (110, 115) of the plurality of open cells may conform to shapes and geometries including, but not limited to: a rhombus, a trapezoid, a square, a rectangle, a parallelogram, a diamond, any equivalent shape or geometry, or any combination or permutation of the aforementioned. In this and other examples, a first pair of opposing (e.g., parallel) segments of the single filament 40 may be arranged with a second pair of opposing (e.g., parallel) segments of the single filament 40 to form the pseudo hooked sections 122 (e.g., four apices 120) of each of the plurality of open cells 110, 115. In other words, a first segment of the single filament 40 may converge with a second segment of the single filament 40 at a first bend or apex (e.g., peak or valley) of the single filament 40 at a first pseudo hooked section 122 of an open cell 110, 115. Further, a third segment of the single filament 40 may converge with a fourth segment of the single filament 40 at a second bend or apex (e.g., valley or peak) of the single filament 40 at a second pseudo hooked section 122 of an open cell 110, 115, with the second bend or apex on an opposite side of the open cell 110, 115 from the first bend or apex. Furthermore, the first segment of the single filament 40 may overlap with the third segment of the single filament 40 at a third pseudo hooked section 122 of an open cell 110, 115, and the second segment of the single filament 40 may overlap with the fourth segment of the single filament 40 to form a fourth pseudo hooked section 122 of an open cell 110, 115. The aforementioned bending and overlapping routine may be repeated indefinitely to form the body of the stent 10.

The plurality of open cells 110, 115 may be differentiated by rows of open cells 110, 115 in which the perimeter and/or area of the open cells 110, 115 varies from row to row. The rows of open cells 110, 115 may extend helically around the tubular scaffold 12 of the stent 10. In other words, the plurality of open cells 110, 115 may include a combination of small open cells 110 and large open cells 115, in which the large open cells have a perimeter and/or area greater than the small open cells 110.

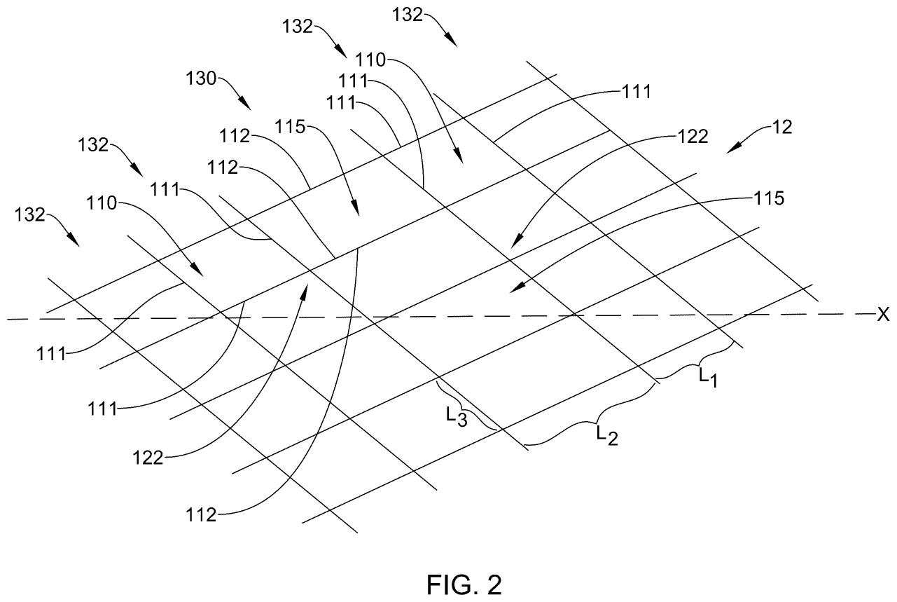

Turning to the schematic depiction of the tubular scaffold 12 shown in FIG. 2, in non-limiting examples, one row 130 of open cells 115 may include large open cells 115 with a greater perimeter and/or area than one or more rows 132 of small open cells 110. The rows of the plurality of open cells 110, 115 may be helically arranged, linearly arranged, or may ascribe to any known pattern, array or arrangement. In one instance, one helical row 130 of open cells 115 may include large open cells 115 with a greater perimeter and/or area than one or more helical rows 132 of small open cells 110. In other words, when viewed from the side of the body of the tubular scaffold 12 of the stent 10, the plurality of open cells 110, 115 may be arrayed in helically extending rows extending helically around the outer circumferential surface of the body of the tubular scaffold 12 of the stent 10. It can be appreciated that the plurality of rows of open cells 110, 115 may extend around the central longitudinal axis of the body of the tubular scaffold 12 of the stent 10 in a helical manner. Whereby the central longitudinal axis of the body of the tubular scaffold 12 of the stent 10 is defined as the axis running through the center of the body of the stent 10 in the longitudinal direction (i.e., along the length and longest dimension of the stent 10). In other non-limiting examples, the plurality of rows of open cells 110, 115 may extend around the central longitudinal axis of the body of the stent in a serpentine pattern (i.e., s-shaped or snake-shaped), a curvilinear pattern, a linear pattern, or any of the equivalent, the like, or any pattern desired.

In yet other non-limiting examples, at least one linear section 111 of each of the plurality of open cells 110, 115 is shared with at least one other linear section 111 of another open cell 110, 115 of each of the plurality of open cells 110, 115. In other words, and in this and other non-limiting examples, the segment of the single filament 40 defining a linear section 111 of one open cell 110, 115 may be the same segment of the single filament 40 defining a linear section 111 of another open cell 110, 115. Furthermore, the segment of the single filament 40 defining a linear section 112 of one open cell 115 may be the same segment of the single filament 40 defining a linear section 112 of another open cell 115.

In some instances, the tubular scaffold 12 may include at least one row of large open cells 115 with a greater perimeter and/or area than at least one row of small open cells 110. In other non-limiting examples, the tubular scaffold 12 may include at least two rows of large open cells 115 with a greater perimeter and/or area than at least one row of small open cells 110. In yet other non-limiting examples, the tubular scaffold 12 may include at least one row of large open cells 115 with a greater perimeter and/or area than the open cells of the remainder of the tubular scaffold 12 (i.e., greater than all of the rows of small open cells 110.

In an alternative embodiment, the tubular scaffold 12 may include open cells 110 all of similar or the same geometry. For example, in examples of the present disclosure, each open cell 110 of the plurality of open cells 110 of the tubular scaffold 12 may all conform to a rhombus shape in the deployed configuration of the stent 10. In this and other examples, the linear sections 111 of the plurality of open cells 110 may all be of the same length. In other non-limiting examples, the linear sections of the plurality of open cells may be of differing or varying lengths. In yet other non-limiting examples, the plurality of open cells may all conform to a trapezoidal shape in which one linear section of an open cell is longer than an opposing linear section of the same open cell. It is further contemplated that this pattern and all patterns contemplated may be extrapolated to the additional open cells of the plurality of cells 110, 115. In other non-limiting examples, the plurality of open cells may all conform to a diamond shape, a square shape, a parallelogram shape, a rectangular shape, a polygonal shape, a triangular shape or any suitable shape desired.

Additionally or alternatively, the size of the plurality of open cells 110, 115, i.e., the area encompassed by the perimeter of the open cell 110, 115, may vary from open cell to open cell. In other words, the large open cells 115 may include at least one side that has a greater length than the length of the sides of the small open cells 110. For instance, the large open cells 115 may include a first pair of opposing sides defined by a long linear section 112 and a second pair of opposing sides defined by a short linear section 111, such that the large open cells 115 form a parallelogram having adjacent sides of different length. Additionally, the small open cells 110 may include a first pair of opposing sides defined by a short liner section 111 and a second pair of opposing sides defined by another short linear section 111. The short linear sections 111 may have a length L1 and the long linear sections 112 may have length L2, wherein the length L2 of the long linear sections 112 is greater than the length L1 of the short linear sections 111. The short linear section 111 of the large open cells 115 may have a length L3. In some instances, the length L3 may be equal to the length L1, such that the small open cells 110 form a rhombus shape. In some instances, the length L3 may be different from the length L1, such that the small open cells 110 form a parallelogram having adjacent sides of different length.

As shown in FIG. 2, the helical row 130 of large open cells 115 may be positioned between a first helical row 132 of small open cells 110 and a second helical row 132 of small open cells 110, in some instances. Thus, the short linear section 111 of the large open cells 115 may be shared with an adjacent small open cell 110. The long linear section 112 of the large open cells 115 may be shared with an adjacent large open cell 115.

Each of the large open cells 115 of the helical row 130 of the large open cells 115 may have a longitudinal extent (i.e., length measured parallel to the central longitudinal axis X) that is greater than a longitudinal extent (i.e., length measured parallel to the central longitudinal axis X) of each of the small open cells 110 of the helical row 132 of the small open cells 110. Thus, the axial distance between opposing ends (e.g., opposing apices 120 or pseudo hooked regions 122) of each of the plurality of large open cells 115 may be greater than the axial distance between opposing ends (e.g., opposing apices 120 or pseudo hooked regions 122) of each of the plurality of small open cells 110.

In some instances, the tubular scaffold 12 may include one or more open cells 110 near the first end 20 of the stent 10 larger or smaller than an open cell 115 near the second end 22 of the stent 10, or near the midpoint of the stent 10 or vice-versa. In other non-limiting examples, the size of the plurality of open cells 110, 115 may vary along the length of the body of the tubular scaffold 12 of the stent 10. In yet other non-limiting examples, each of the open cells 110, 115 of the plurality of open cells may possess similar, equal or substantially equal sizes.

In yet other non-limiting examples, the size of the open cells 110, 115 of the plurality of open cells may vary within each row of the open cells 110, 115. In other words, an open cell 115 may possess greater size (i.e., the area encompassed by the perimeter of the open cell) than an adjacent open cell 110 in the same row. In yet other non-limiting examples, an open cell 110 may be of a smaller perimeter (i.e., a smaller size) than an adjacent open cell 115 in the same row. In other non-limiting examples, a pattern of smaller and larger open cells 110, 115 may repeat along the row, repeat alternately along the row, repeat intermittently along the row, or ascribe to any pattern, routine, scheme or variation desired. In yet other non-limiting examples, the size of the open cells 110, 115 of the plurality of open cells may vary between each row of the open cells 110, 115. For instance, and by non-limiting example, an open cell 115 of a first row may possess greater size (i.e., the area encompassed by the perimeter of the open cell) than an open cell 110 present in a different row, or vice-versa. In yet other non-limiting examples, an open cell of a first row may be of greater size than an open cell of a second row but smaller than an open cell of a third row. It is further contemplated that any pattern or variance of open cell 110, 115 sizes, areas, perimeters, lengths and any combination or permutation of open cell 110, 115 sizes, areas, perimeters, and lengths may be dependent on the row the open cell 110, 115 resides in and may be employed as is suitable for the purposes of the present disclosure.

As described herein, the single filament 40 may shaped such that at least one or more segments of the single filament 40 changes direction after forming at least one pseudo hooked section 122 at the apices 120 of each of the plurality of open cells 110, 115. In other words, the single filament 40 may shaped and arranged such that one apex 120 (e.g., peak or valley) of the single filament 40 overlaps with another apex 120 (e.g., valley or peak) of the single filament 40 to form a pseudo hooked section 122 of an open cell 110, 115. It can be appreciated that the single filament 40 may be shaped and arranged to form additional open cells 110, 115 throughout the body of the tubular scaffold 12 of the stent 10. The single filament 40 may be shaped and arranged such that it forms at least two linear sections 111 and at least two pseudo hooked sections 112 of each of the plurality of open cells (110, 115). The single filament 40 may be shaped and arranged such that a row of open cells (e.g., a helical row of large open cells 115) contains open cells 115 of a larger perimeter and/or area than open cells 110 in an adjacent row of open cells (e.g., a helical row of small open cells 110). This pattern may repeat, alternate, or follow any pattern desired.

As noted above, the pseudo hooked sections 122 of the open cells 110, 115 may be formed by longitudinally overlapping bends of the filament 40 (i.e. overlapping a peak 50 of the single filament 40 in a first circumferential row of the undulating filament 40 with a valley 52 of the single filament 40 in an adjacent circumferential row of the undulating filament 40), thus forming a pseudo hooked section 122 at an apex 120 of an open cell 110, 115. This formation routine may be extrapolated throughout the entirety of the tubular scaffold 12 of the stent 10 to form the plurality of open cells 110, 115 by the single filament 40.

The segments of the single filament 40 may be bent to a desired radius of curvature at the apex 120 to form the pseudo hooked section 122. In this and other examples, the radius of curvature of the bent segments (peaks 50 and/or valleys 52) of the single filament 40 may be about 0.05 mm or less, about 0.10 mm or less, about 0.25 mm or less, about 0.35 mm or less, or about 0.5 mm or less, for example.

Additionally or alternatively, the thickness of the filament 40 may vary along the length of the filament 40. In other words, the filament 40 may be thinner (e.g., have a smaller diameter) at one end of the stent 10 (e.g., the first end 20 or the second end 22), but thicker (e.g., have a larger diameter) at the other end of stent 10 (e.g., the second end 22 or the first end 20), or vice-versa. In yet other non-limiting examples, the thickness (e.g., diameter) of the filament 40 may change or transition between multiple thicknesses over the length of the filament 40. In yet other non-limiting examples, the filament 40 may be hollow, partially hollow, intermittently hollow, substantially hollow, radiopaque, partially radiopaque, substantially radiopaque, or intermittently doped with radiopaque material.

Additionally or alternatively, in some instances, segments of the single filament 40 may intertwine with one another at one or more crossing points by intersecting helically, through twisting, or through other known methods of intertwining. Additionally or alternatively, a second filament may be incorporated into construction of the tubular scaffold 12 of the stent 10. The second filament may follow the same pattern as the first filament, or may be shaped into a different pattern, or may be arranged along a portion of the first filament, for example. Additionally or alternatively, a third filament may be incorporated into construction of the tubular scaffold 12 of the stent 10. In yet other non-limiting examples, four or more filaments may be incorporated into construction of the tubular scaffold 12 of the stent 10.

As described herein, each of the plurality of small open cells 110 of the tubular scaffold 12 of the stent 10 may include four pseudo hooked sections 122, with one formed at each apex of the small open cell 110, and two pairs of opposing (e.g., parallel) linear sections 111. Each of the plurality of large open cells 115 of the tubular scaffold 12 of the stent 10 may include four pseudo hooked sections 122, with one formed at each apex of the large open cell 115, a first pair of opposing (e.g., parallel) long linear sections 112 and a second pair of opposing (e.g., parallel) short linear sections 111. In yet other non-limiting examples, each of the plurality of open cells 110, 115 of the tubular scaffold 12 of the stent 10 may only include a single pseudo hooked section 122, only include two pseudo hooked sections 122, or only include three pseudo hooked sections at the apices 120, wherein the remaining apices 120 may be formed by cross-over points of the single filament 40 and/or true hooked sections of the single filament 40. Other combinations and permutations of the aforementioned are contemplated by the disclosure and well within the ambit of one of ordinary skill in the art.

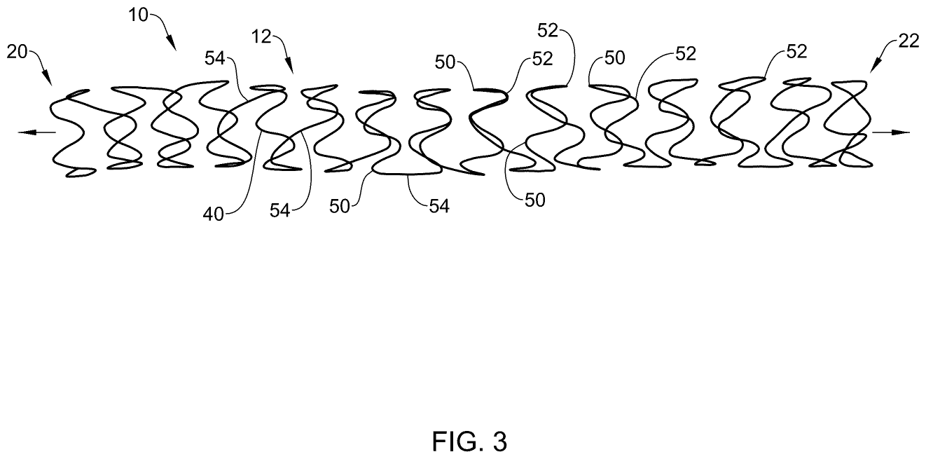

FIG. 3 illustrates the tubular scaffold 12 of the stent 10 in an axially elongated configuration (e.g., non-equilibrium configuration) in which an axial force (shown by arrows) is applied to the tubular scaffold 12 to elongate the tubular scaffold 12 away from its equilibrium configuration shown in FIG. 1. Thus, the axially elongated configuration shown in FIG. 3 may be considered a configuration in which the tubular scaffold 12 is subjected to an external axial force deforming the tubular scaffold from its equilibrium configuration. FIG. 3 is provided to illustrate that the tubular scaffold 12 may be formed of a plurality of circumferential rings of the single filament 40 in which a connecting segment 54 of the filament 40 interconnects adjacent circumferential rings of the tubular scaffold 12. Each circumferential ring may be formed of the filament 40 in which the filament 40 undulates to form a plurality of peaks 50 oriented toward the first end 20 of the tubular scaffold 12 and valleys 52 oriented toward the second end 22. In the axially elongated configuration, the peaks 50 of one circumferential ring may move away from the valleys 52 of an adjacent circumferential ring since the peaks 50 are not intertwined or interlocked with the valleys 52, thereby providing an axial gap therebetween. Likewise, the valleys 52 of one circumferential ring may move away from the peaks 50 of an adjacent circumferential ring since the peaks 50 are not intertwined or interlocked with the valleys 52, thereby providing an axial gap therebetween. The separation of the peaks 50 from the valleys 52 may allow the stent 10 to bend within an anatomical site without kinking or buckling the stent 10.

The filament 40 of the tubular scaffold 12 of the stent 10 may be formed from one or more suitable materials. Example suitable materials include, but are not limited to, metals, metal alloys, shape memory alloys, polymers, nickel-titanium alloys, cobalt-chromium-nickel-molybdenum alloys, and/or other suitable materials enabling the tubular scaffold 12, and thus the stent 10, to be radially expanded into a shape when positioned at a target site. In some instances, the material may be selected to enable the stent 10 to be removed with relative ease as well. In some examples, the filament 40 may be formed from alloys such as, but not limited to, nitinol and/or Elgiloy®.

The body of the tubular scaffold 12 of the stent 10 may include a radially constrained configuration (e.g., unexpanded or delivery configuration), a radially unconstrained and expanded configuration (e.g., expanded or deployed configuration), and a partially constrained/unconstrained transition between the constrained and unconstrained configurations.

In various embodiments, the tubular scaffold 12 of the stent 10 may be partially or fully covered, uncovered, coated, or a combination thereof. Various stent embodiments described herein may include a full or partial covering, coating, or other membrane over an interior surface of the tubular scaffold 12 and/or over an exterior surface of the tubular scaffold 12. For example, a covering (e.g., coating, or other membrane) may comprise silicone, a polymer, or a combination thereof. For example, a cover may comprise polyurethane, polytetrafluoroethylene, expanded polytetrafluoroethylene, polyvinylidene fluoride, an aromatic polycarbonate-based thermoplastic urethane, and/or other like materials. A covering may include ingrowth promoting materials for interfacing with tissue. A covering may be applied by dip coating, roll coating, painting, spraying, other known disposition method, or a combination thereof. A covering, coating, or other membrane may inhibit tissue growth and/or minimize fluid leakage from within and/or without the stent.

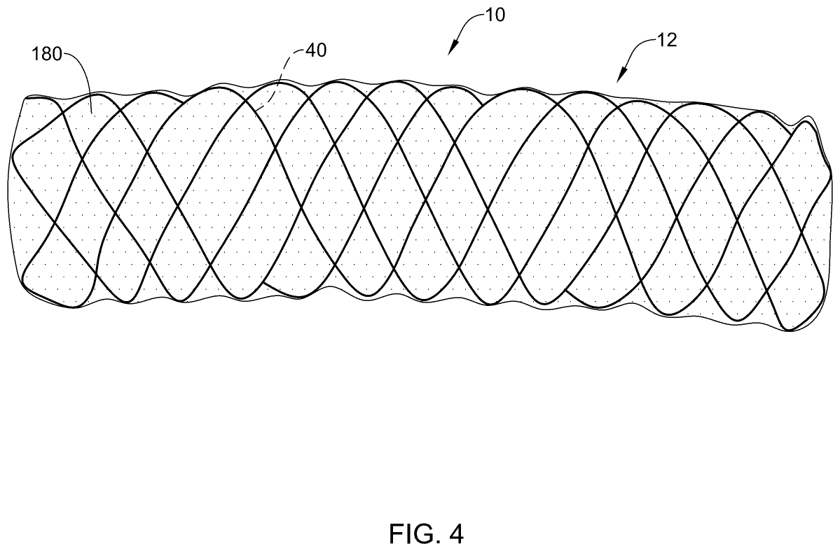

FIG. 4 depicts an illustrative stent of the present disclosure. As shown in FIG. 4, a covering 180 is applied to the tubular scaffold 12 of the stent 10. The covering 180 may be any desired covering, including but not limited to a coating, a wrap, a sleeve, a sheath, a circumferential sleeve, a circumferential sheath, formed of any desired material.

Applying a coating and/or covering is advantageous for myriad reasons. For instance, and by non-limiting example, applying a coating and/or covering may preclude tissue ingrowth within the lumen of the stent 10. By limiting tissue ingrowth, the stent 10 is less likely to become occluded with organic matter and allows proper perfusion and flow of biological fluids through the stent 10 during its life of implantation. Additional advantages conferred by a coated and/or covered stent include improved conformability of the body of the stent 10 as it is delivered through the tortuous anatomy of a subject or patient's body. Applying a coating and/or covering also increases the durability and effective lifetime of the stent, which in turn reduces the overall costs associated with manufacture and implementation.

A covering 180 (e.g., coating), when applied to the tubular scaffold 12 of the stent 10, may be applied to any suitable portion of the stent 10. The covering 180 (e.g., coating) may be applied to an entirety of the body of the tubular scaffold 12 of the stent 10, but this is not required and the covering 180 (e.g., coating) may be applied to only a portion of the tubular scaffold 12 of the stent 10 that is less than the entirety of the stent 10. For example, the covering 180 may not extend an entire length of the stent 10, leaving portions of the tubular scaffold 12 uncovered and devoid of the covering 180.

Some examples of suitable materials for the stents 10 and/or covering 180 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

The stent 10 may be a self-expanding stent (SES) or a self-expanding metallic stent (SEMS), meaning that the stent 10 may automatically expand into an expanded configuration (e.g., deployed configuration) once any constraints preventing expansion have been removed. In some instances, the stent 10 may not be a self-expanding stent, and thus may rely upon an inflatable balloon or other expandable structure to cause the stent 10 to expand from a collapsed configuration for delivery to its expanded configuration for deployment in a body lumen.

The stent 10 may be configured to bend into a U-shape and/or an S-shape without kinking. This provides an advantageous benefit in deployment of the stent 10 as stent 10 is able to comply with the tortuous anatomy of a patient or subject as the stent 10 is guided toward its terminal destination of deployment within a patient or subject. In other words, when stent 10 is deployed, for instance through a vessel or other body lumen, the stent 10 is able to bend along the curvature of the body lumen as the stent 10 is positioned through the body lumen of a patient or subject without causing undue trauma to the tissue of the patient or subject (e.g., lumens, vessels, organs, organ tissue, lumen walls, vessel walls, etc.). Owing to the flexibility of the filament 40 and flexibility of covering 180 (e.g., coating), the stent 10 is also able to revert into its radially expanded shape upon deployment, and maintain patency of a lumen, vessel, duct or the like at its terminal destination of deployment. In other words, the stent 10 may be delivered in a straight configuration for delivery into a patient through a device such as a catheter. As the stent 10 is navigated through the body lumen of a patient or subject, the stent 10 is able to bend with the curves of the body lumen of the patient or subject without causing undue trauma to the body lumen of the patient or subject. Upon reaching the desired area of treatment, the stent 10 may revert to its original radially expanded shape or configuration, or may further conform to the geometry of the desired area of treatment. For instance, and by non-limiting example, stent 10 may conform to a bend of the bile duct and/or pancreatic duct while maintaining patency in the bile duct or pancreatic duct.

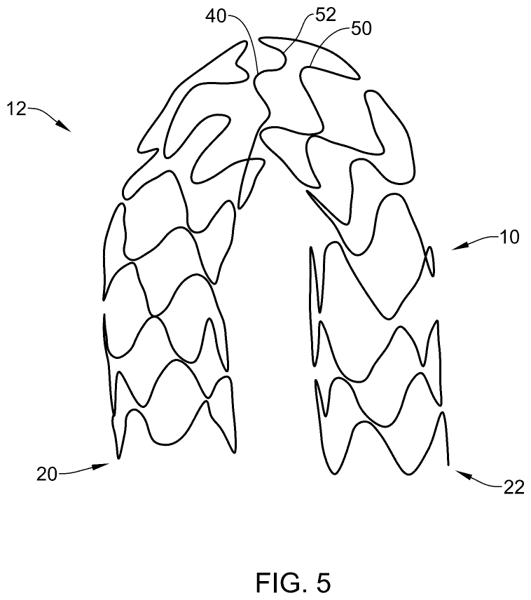

FIG. 5 depicts an illustrative stent 10 of the present disclosure constrained in a bent configuration. As shown in FIG. 5, when the stent 10 is constrained in a bent configuration away from its equilibrium configuration, the peaks 50 may move away from the valleys 52 to facilitate bending the stent 10 without kinking. Thus, since the peaks 50 are not intertwined or interlocked with the valleys 52, the undulating filament rows may separate from one another at least on a radially convex side (i.e., outer curvature) of the bent stent 10. The separation of the peaks 50 from the valleys 52 may allow the stent 10 to lengthen around the outer radius of the curvature of the bend without kinking or buckling along the inner radius of the bend.

Methods are also contemplated by the present disclosure. In a non-limiting example method, a single filament 40 may be shaped to form a tubular scaffold having a plurality of open cells 110, 115 throughout the tubular scaffold; whereby apices (e.g., peaks 50 and valleys 52) of the single filament 40 may be overlapped in a pseudo hooked configuration to form two opposing pairs of linear sections 111 of each of the plurality of open cells 110, 115 converging at apices of the open cells 110, 115; whereby at least two apices (e.g., a peak 50 and a valley 52) of the single filament 40 overlap to form a pseudo hooked section 112 at the apices of each of the plurality of open cells 110, 115.

Additionally or alternatively, methods are also contemplated in the same manner as above but may further include additional filaments. In other words, more than one filament may be shaped to form at least a portion of the plurality of open cells 110, 115 throughout the body of the tubular scaffold 12; whereby multiple filaments may be combined to form each of the plurality of open cells 110, 115; whereby the pseudo hooked sections 122 at the apices 120 of each of the plurality of open cells 110, 115 in formed by overlapping the multiple filaments.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent comprising:
a tubular scaffold extending from a first end to a second end;
the tubular scaffold formed of a single filament;
wherein the single filament is shaped to form a plurality of open cells throughout the tubular scaffold, each of the plurality of open cells defined by a plurality of apices of the single filament;
wherein at least one of the plurality of apices includes a pseudo hooked segment wherein a first bend of the single filament longitudinally overlaps a second bend of the single filament.

2. The stent of claim 1, wherein the plurality of open cells includes a first helical row of large open cells, a first helical row of small open cells, and a second helical row of small open cells; and
wherein the first helical row of large open cells is positioned between the first helical row of small open cells and the second helical row of small open cells.

3. The stent of claim 2, wherein each of the large open cells has a greater perimeter and/or area than each of the small open cells.

4. The stent of claim 2, wherein each of the large open cells has a longitudinal extent greater than a longitudinal extent of each of the small open cells.

5. The stent of claim 2, wherein each of the large open cells has a parallelogram shape having four apices.

6. The stent of claim 5, wherein each of the four apices of the large open cells includes a pseudo hooked segment.

7. The stent of claim 5, wherein each of the small open cells has a rhombus shape having four apices.

8. The stent of claim 7, wherein each of the four apices of the small open cells includes a pseudo hooked segment.

9. A stent comprising:
a tubular scaffold extending from a first end to a second end;
the tubular scaffold formed of a single filament defining a plurality of open cells having apices;
wherein the single filament forms a plurality of circumferential rings, wherein each circumferential ring is formed of the single filament in which the single filament undulates to form a plurality of peaks oriented toward the first end and a plurality of valleys oriented toward the second end;
wherein each of the apices of the plurality of open cells includes a pseudo hooked region in which the plurality of peaks of a first circumferential ring of the plurality of circumferential rings longitudinally overlap the plurality of valleys of a second circumferential ring of the plurality of circumferential rings.

10. The stent of claim 9, wherein in an axially elongated, non-equilibrium configuration, the plurality of peaks of the first circumferential ring are axially spaced apart from the plurality of valleys of the second circumferential ring with an axial gap therebetween.

11. The stent of claim 9, wherein each of the plurality of open cells is formed as a parallelogram shape defined by two pairs of opposing linear sections of the filament and pseudo hooked regions of the filament at each apex of the plurality of open cells.

12. The stent of claim 9, wherein the plurality of open cells includes a first helical row of large open cells, a first helical row of small open cells, and a second helical row of small open cells;
wherein the first helical row of large open cells is positioned between the first helical row of small open cells and the second helical row of small open cells.

13. The stent of claim 12, wherein each of the large opens cells shares a side with one of the small open cells.

14. The stent of claim 12, wherein each of the large open cells has a longitudinal extent greater than a longitudinal extent of each of the small open cells.

15. A stent comprising:
a tubular scaffold extending from a first end to a second end;
the tubular scaffold formed of a single filament;
wherein the single filament is shaped to form a plurality of open cells throughout the tubular scaffold;
wherein each of the plurality of open cells is defined by at least two pairs of opposing linear sections of the single filament and at least two pseudo hooked segments of the single filament;
wherein the single filament is bent to form the at least two pairs of opposing linear sections and the at least two pseudo hooked segments, and
wherein a first bend of the single filament longitudinally overlaps a second bend of the single filament at each pseudo hooked segment.

16. The stent of claim 15, wherein each pseudo hooked segment includes a peak of the single filament in a first circumferential ring of the tubular scaffold longitudinally overlapping a valley of the single filament in a second circumferential ring of the tubular scaffold.

17. The stent of claim 15, wherein the plurality of open cells are arranged in a plurality of helical rows extending helically around the tubular scaffold, wherein at least one helical row of the plurality of helical rows comprises a plurality of open cells with a larger perimeter than the plurality of open cells of at least one other helical row of the plurality of helical rows.

18. The stent of claim 17, wherein each of the plurality of open cells has a parallelogram shape having four apices.

19. The stent of claim 18, wherein each of the four apices of the open cells includes a pseudo hooked segment.

20. The stent of claim 15, wherein each of the plurality of open cells conforms to a parallelogram shape.
